(19) United States
(12) Patent Application Publication
(10) Pub. No.: US 2026/0160747 A1
(43) Pub. Date: Jun. 11, 2026

(54) COAL-ROCK-GANGUE SORTING METHOD AND SYSTEM BASED ON MULTI-SOURCE DATA FUSION ANALYSIS

(71) Applicant: Shandong University, Jinan (CN)

(72) Inventors: Zhenhao XU, Jinan (CN); Tengfei Yu, Jinan (CN); Peng Lin, Jinan (CN); Shucai Li, Jinan (CN); Yanfei Lou, Jinan (CN); Tao Han, Jinan (CN); Shenke Du, Jinan (CN)

(73) Assignee: Shandong University, Jinan (CN)

(21) Appl. No.: 19/183,386

(22) Filed: Apr. 18, 2025

(30) Foreign Application Priority Data
Dec. 4, 2024 (CN) 2024117707831

(51) Int. Cl. G01N 33/24; G01N 3/06

(52) U.S. Cl. CPC G01N 33/24; G01N 3/068; G01N 2203/0641; G01N 2203/0694

(57) ABSTRACT

The present invention discloses a coal-rock-gangue sorting method based on multi-source data fusion analysis, comprising: conducting identical load-bearing tests on rock blocks and coal blocks, monitoring strain responses and heat released during loading, performing a spectral analysis on the different categories of coal, rock, correlating different the strain responses, release heats, and absorption peak features with corresponding categories of coal, rock, and gangue, to build separately a strain-based, a heat-based and a spectral-based multi-classification model, classifying coal-rock-gangue mixtures by independently employing the multi-classification models, fusing sorting results of the three multi-classification models, and determining a final sorting result. The present invention effectively addresses the uncertainties caused by single-method approaches by performing deep decision fusion by integrating differences in apparent features, physical properties, and compositional components of coal-rock-gangue, and significantly enhances the accuracy and precision of the sorting result.

### CROSS-REFERENCE TO RELATED APPLICATIONS

The present invention claims priority benefits to Chinese Patent Application number 202411770783.1, entitled “A Coal-rock-gangue Sorting Method and System Based on Multi-source Data Fusion Analysis”, filed on 4 Dec. 2024, with the China National Intellectual Property Administration (CNIPA), the entire contents of which are incorporated herein by reference.

### TECHNICAL FIELD

The present invention pertains to the field of coal-rock-gangue sorting, specifically relating to a coal-rock-gangue sorting method and system based on multi-source data fusion analysis.

### BACKGROUND

The statements in this section merely provide background information related to the present invention and are not necessarily prior art.

In coal mining, efficient and accurate coal-rock-gangue identification is a critical prerequisite for enhancing economic efficiency and reducing equipment wear. During actual coal extraction, complex coal-rock-gangue mixtures are frequently encountered. The presence of intermixed coal, rock, and gangue degrades coal quality since rock and gangue are non-combustible materials. If not separated, the combustion of coal containing rock and gangue produces excessive ash, reduces heat value, and lowers combustion efficiency. Additionally, the presence of rock and gangue significantly increases transportation costs. Therefore, achieving precise and efficient coal-rock-gangue sorting is an essential step in coal mining and processing.

The existing coal-rock-gangue sorting methods and their associated challenges are as follows: I) manual sorting: the classification is performed based on color, shape, and weight, relying on qualitative judgments made by human operators. However, this method is labor-intensive, has low sorting efficiency, and often results in incomplete sorting. II) Dense-medium sorting: this method utilizes differences in density between coal and gangue for sorting; however, the process is complex and cumbersome. Additionally, due to the diversity of coal categories, it is difficult to ensure a significant density difference between all coal, rock, and gangue materials, leading to reduced sorting accuracy and efficiency. III) Radiation-based detection: this method leverages differences in the reflection and absorption of γ-rays and X-rays by coal and gangue for identification. However, the γ-rays and X-rays emit strong radiation, posing health hazards and safety risks. And, IV) machine vision recognition: this technique captures image data of coal, rock, and gangue and applies machine learning algorithms to analyze gray level, texture, and shape features for identification. However, this method relies on acquiring clear and complete images. In actual conveyor belt operations, coal-gangue mixtures are often affected by dust and poor lighting conditions, significantly increasing recognition difficulty.

### SUMMARY

To overcome the limitations of the existing technologies, the present invention provides a coal-rock-gangue sorting method and system based on multi-source data fusion analysis, which integrates differences in apparent features, physical properties, and compositional components among coal, rock, and gangue to enable deep-level decision fusion, to achieve a high-precision sorting of coal-rock-gangue mixtures, thereby enhancing coal-rock-gangue sorting technology and promoting the advancement of the coal industry.

To achieve the aforementioned objectives, one or more embodiments of the present invention provide the following technical solutions.

In the first aspect, the present invention proposes a coal-rock-gangue sorting method based on multi-source data fusion analysis, comprising:
collecting different categories of coal, rock, and gangue, and processing the collected different categories of coal, rock, and gangue into rock blocks and coal blocks of uniform size;
conducting identical load-bearing tests on the processed rock blocks and coal blocks, monitoring strain responses of the rock blocks and the coal blocks in the tests, correlating different the monitored strain responses with corresponding categories of coal, rock, and gangue, and building a strain-based multi-classification model;
monitoring a heat released from the coal blocks and the rock blocks during the load-bearing tests, correlating emitted heat under identical size and loading conditions with the corresponding categories of coal, rock, and gangue, and building a heat-based multi-classification model;
performing a spectral analysis on the different categories of coal, rock, and gangue to identify spectral absorption peak features, correlating the identified spectral absorption peak features with the corresponding categories of coal, rock, and gangue, and building a spectral-based multi-classification model;
employing the strain-based, heat-based, and spectral-based multi-classification models to independently classify coal-rock-gangue mixtures, and by fusing sorting results of the strain-based, heat-based, and spectral-based multi-classification models, determining and outputting a final sorting result.

In a further technical solution, the strain-based multi-classification model is trained using a first neural network, wherein input parameters comprise a total strain of the blocks and strain values recorded at different time intervals, while an output parameter is a probability distribution of a corresponding type of coal, rock, or gangue.

In a further technical solution, the heat-based multi-classification model is trained using a second neural network, wherein input parameters comprise a total heat released, a heat release rate, and an average temperature of the blocks under the identical size and load-bearing conditions, while an output parameter is the probability distribution of the corresponding type of coal, rock, or gangue.

In a further technical solution, the spectral-based multi-classification model is trained using a third neural network, wherein input parameters comprise the absorption peak features from different blocks, while an output parameter is the probability distribution of the corresponding type of coal, rock, or gangue.

In a further technical solution, determining and outputting the final sorting result by fusing the sorting results of the strain-based, heat-based, and spectral-based multi-classification models, specifically comprising: applying a weighted averaging method to three the sorting results, selecting a category with the highest probability after the weighted averaging as the final sorting result, and separately conveying and transporting the separated coal and rock.

In a further technical solution, a weighting process is dynamic weighting based on an uncertainty of prediction of a fusion model. The uncertainty is quantified by using an entropy value, and weights are assigned according to entropy values. The entropy value is computed using the following formula:

$$H ( p ) = - {\sum }_{i = 1}^{C} {P}_{i} log ( {P}_{i} ) ,$$

where, p denotes the probability distribution of the categories predicted by the model, C denotes a total number of the categories, and Pᵢ denotes a probability that the model classifies a sample into category i.

In a further technical solution, the weighted averaging method is applied based on the assigned weights. The category with the highest probability after weighted averaging is selected as the final sorting result. The calculation formula of the weight Wₘ is as follows:

$${W}_{m} = \frac{1 / {H}_{m}}{{\sum }_{1}^{M} 1 / {H}_{k}} ,$$

where, Hₘ denotes the entropy value of the m^{th} model for a current sample, M denotes a total number of classification models, k denotes the k^{th} classification model, Wₘ denotes the weight assigned to the m^{th} model, and Hₖ denotes the entropy value of the k^{th} model for the current sample.

Letting a probability predicted by the model m be

$${p}_{m} = \{ {p}_{1}^{m} , {p}_{2}^{m} , {p}_{3}^{m} , … , {p}_{C}^{m} \} ,$$

then the weighted final output probability is:

$${p}_{final} = {\sum }_{m = 1}^{M} {W}_{m} * {p}_{m} .$$

In the second aspect, the present invention further provides a coal-rock-gangue sorting system based on multi-source data fusion analysis, comprising:
a sample collection and processing module, configured to: collect different categories of coal, rock, and gangue and process the collected coal, rock, and gangue into rock blocks and coal blocks of uniform size;
a strain-based multi-classification model building module, configured to performs identical load-bearing tests, monitor strain responses of the coal blocks and the rock blocks, and build a strain-based multi-classification model;
a heat-based multi-classification model building module, configured to monitor heat released from the coal blocks and the rock blocks during the load-bearing tests and build a heat-based multi-classification model;
a spectral-based multi-classification model building module, configured to analyze spectral absorption peak features of different coal, rock, and gangue samples and build a spectral-based multi-classification model; and
a fusion sorting module, configured to integrate sorting results from the strain-based, heat-based, and spectral-based multi-classification models to determine a final sorting result.

In the third aspect, the present invention provides a computer-readable storage medium, the computer-readable storage medium is non-transitory and stores a computer program thereon that when executed by a processor, causes the processor to implement steps of a coal-rock-gangue sorting method based on multi-source data fusion analysis as described in the first aspect.

In the fourth aspect, the present invention provides a computer device, comprising a memory, a processor, and a computer program stored in the memory and executable on the processor; when the computer program is executed by the processor, causes the computer device to implement steps of a coal-rock-gangue sorting method based on multi-source data fusion analysis as described in the first aspect.

One or more of the above technical solutions have the following beneficial effects:

According to the present invention, differences in apparent features, physical properties, and compositional components among coal, rock, and gangue are integrated, wherein in terms of the apparent features, by considering the impact of dust in the sorting environment, non-contact strain detection is used instead of image recognition for sorting; in terms of the physical properties, coal and rock exhibit differences in thermodynamic properties, molecular structure, and mechanical properties, and they release varying amounts of heat when compressed, so that heat release temperature is selected as a key physical parameter for sorting; in terms of the compositional components, a compositional composition is a fundamental distinguishing factor among different materials, since the coal, rock, and gangue have distinct compositional components, spectral analysis is employed for quantitative composition analysis. Moreover, this method integrates multiple sorting techniques through deep decision fusion, which significantly enhances the accuracy and precision of coal-rock-gangue sorting, effectively addressing the uncertainties caused by single-method approaches. By combining strain, heat, and spectral data, sorting is performed from three distinct perspectives: the apparent features, the physical properties, and the compositional differences. Compared to density-based sorting or manual sorting, this method is easier to implement, features a simplified process, and demonstrates higher practical applicability.

According to the present invention, fusing strain-based, heat-based, and spectral-based multi-classification models that represent different feature levels, are easy to implement, and exhibit strong complementarity, allowing for enhanced fusion performance in deep decision-making processes. Additionally, during model fusion, entropy-based dynamic weighting is applied to quantify prediction uncertainty. This allows for flexible adjustment of model weights based on uncertainty levels, thereby improving both the adaptability and classification accuracy of the system.

The present invention enables precise sorting of coal, rock, and gangue, thereby increasing the proportion of marketable coal while reducing processing and transportation costs. Additionally, this method optimizes resource utilization efficiency, further contributing to cost-effectiveness and sustainability in coal processing operations.

### BRIEF DESCRIPTION OF THE DRAWINGS

The drawings comprised in this specification form an integral part of the present invention and are provided to further illustrate the invention. The illustrative embodiments and their descriptions are intended to explain the invention and should not be construed as improper limitations on its scope.

FIG. 1 is a flowchart illustrating the coal-rock-gangue sorting method in an embodiment of the present invention.

### DETAILED DESCRIPTION

It should be noted that the following detailed descriptions are illustrative and are intended to provide further explanation of the present invention. Unless otherwise specified, all technical and scientific terms used herein have the same meaning as commonly understood by those skilled in the relevant field.

It should be noted that the terminology used herein is intended solely for describing specific embodiments and is not intended to limit the exemplary embodiments of the present invention. As used in this specification, unless explicitly stated otherwise in the context, singular forms should be interpreted to comprise plural forms as well. Additionally, it should be understood that whenever the terms “comprises” and/or “comprising” are used in this specification, they indicate the presence of specific features, steps, operations, devices, components, and/or combinations thereof, but do not preclude the existence or addition of other features, steps, operations, devices, components, and/or combinations thereof.

Where there is no conflict, the embodiments of the present invention and the features described in different embodiments may be combined.

### Example 1

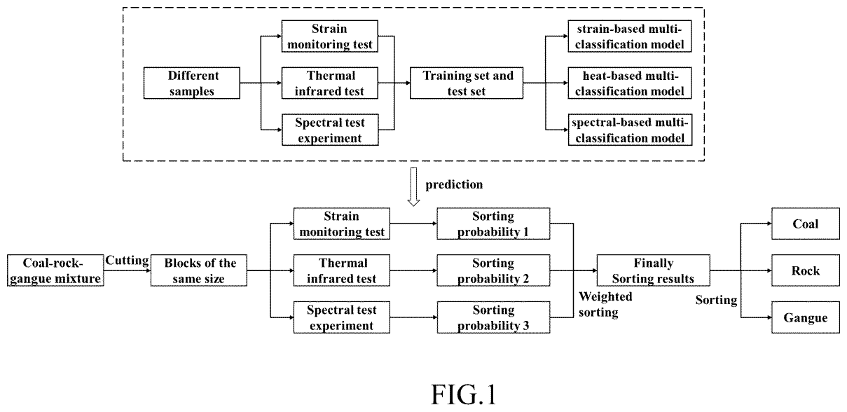

As illustrated in FIG. 1, the present example discloses a coal-rock-gangue sorting method based on multi-source data fusion analysis, comprising the following steps:

Step 1: Collecting different categories of coal, rock, and gangue, and processing them into rock blocks and coal blocks of uniform size.

In the present example, the different categories of coal comprise fat coal, lignite, coking coal, gas-fat coal, gas coal, and anthracite. The different categories of rock comprise granite, diabase, sandstone, limestone, quartz sandstone, quartzite, plagioclase, and shale, which are commonly found in coal mining areas. The different categories of gangue comprise lignite gangue and bituminous coal gangue.

Step 2: Conducting identical load-bearing tests on the rock blocks and coal blocks, monitoring the strain responses of the coal blocks and the rock blocks, and performing correlation analysis to associate different strain values with their corresponding coal, rock, and gangue categories, thereby building a strain-based multi-classification model.

Correlation analysis involves building a nonlinear complex mapping relationship between strain values and the respective coal, rock, and gangue categories.

In the present example, a three-dimensional (3D) non-contact strain monitoring device is used to measure the strain responses of the coal blocks and the rock blocks. Before conducting the experiment, the 3D non-contact strain monitoring device must be calibrated. The calibration process comprises checking the optical system and calibration points of the instrument and ensuring that the instrument remains parallel and stable relative to the specimen surface.

The load-bearing test consists of applying an identical axial compressive force to rock blocks and coal blocks of the same size. The 3D non-contact strain monitoring device is used to capture and record both the total strain from the start of loading to the end of loading, as well as the strain values at different time intervals throughout the process.

The strain-based multi-classification model is trained using a neural network. Specifically, the neural network consists of an input layer, hidden layers, and an output layer. The input parameters comprise the total strain of the specimen and strain values recorded at different time intervals. The output parameter is the probability distribution of the sample belonging to coal, rock, or gangue.

The output layer consists of a fully connected layer and a Softmax layer. For a multi-class classification task with C categories, the fully connected layer produces an output Z={Z₁, Z₂, Z₃, . . . , Z_{C}}, Zᵢ representing the un-normalized score for category i. The Softmax layer then maps the un-normalized scores into a probability distribution, where the probability for each category Pᵢ is computed using the following formula:

$${P}_{i} = \frac{{e}^{{Z}_{i}}}{{\sum }_{j = 1}^{c} {e}^{{Z}_{j}}} ,$$

In this formula, Pᵢ denotes the probability that the input sample x belongs to category i,

$${\sum }_{j = 1}^{c} {e}^{{Z}_{j}}$$

denotes the sum of the exponentiated unnormalized scores across all categories, and C denotes the number of categories, and Zⱼ denotes the unnormalized score of category j.

The training process of the neural network involves the following steps: collecting total strain values and strain data at different time intervals from test samples, building a dataset by associating this information with corresponding coal, rock, and gangue categories, and then splitting the dataset into training and test sets. During training, a cross-entropy loss function is utilized to minimize classification errors. In the evaluation phase, classification accuracy, precision, recall, and F1-score are employed as key performance metrics. The model is retained only if it meets the predefined classification accuracy requirements.

Step 3: Monitoring the heat released from the coal blocks and the rock blocks during the loading, correlating the heat released under the same size and loading conditions with the corresponding coal, rock block and gangue categories, and build a heat-based multi-classification model;

The correlation analysis is to build a non-linear complex mapping relationship between the released heat and the coal, rock mass, and gangue categories.

In the present example, the heat released during loading is monitored using an infrared thermal imager. The infrared thermal imager should have high resolution and sensitivity to ensure the accuracy of the measured temperature changes; before starting the test, the infrared thermal imager is aimed at the test blocks to ensure that its field of view covers the entire surface of the test blocks.

The thermal imaging test is specific to record the total amount of heat released, the rate of heat release, and the average temperature of each specimen during loading.

The thermal multi-classification model is trained using a neural network, specifically, the neural network comprises an input layer, a hidden layer, and an output layer, with the input parameters being the total amount of heat release, the rate of heat release, and the average temperature of the specimen blocks of the same size and the same loading conditions, and the output parameter being the probability of the corresponding coal, rock, or gangue type.

The output layer consists of a fully connected layer and a Softmax layer. For a multi-class classification task with C categories, the fully connected layer produces an output Z={Z₁, Z₂, Z₃, . . . , Z_{C}}, Zᵢ representing the un-normalized score for category i. The Softmax layer then maps the un-normalized scores into a probability distribution, where the probability for each category Pᵢ is computed using the following formula:

$${P}_{i} = \frac{{e}^{{Z}_{i}}}{{\sum }_{j = 1}^{c} {e}^{{Z}_{j}}} ,$$

In this formula, Pᵢ denotes the probability that the input sample x belongs to category i,

$${\sum }_{j = 1}^{c} {e}^{{Z}_{j}}$$

denotes the sum of un exponentiated unnormalized scores across all categories, and C denotes the number of categories, and Zⱼ denotes the unnormalised score of category j.

The neural network training process involves building a dataset based on the total amount of heat released, heat release rate, and average temperature measured from test specimens of identical size and load application conditions, along with their corresponding coal, rock, or gangue categories. The dataset is then split into a training set and a test set. The model is trained on the training set using cross-entropy loss as the loss function. Performance evaluation is conducted on the test set, where classification metrics such as accuracy, precision, recall, and F1-score are assessed. Once the model meets the required classification accuracy, the trained multi-class heat classification model is saved.

Step 4: Testing and identifying spectral absorption peaks of different categories of coal, rock, and gangue, correlating different absorption peak features with corresponding categories of coal, rock, or gangue, and building a spectral-based multi-classification model;

Correlation analysis is to build a nonlinear complex mapping of absorption peak features to coal, rock, and gangue categories.

In the present example, different categories of coal, rock, and gangue are tested using near-infrared spectroscopy to analyze the absorption peak features. The wavelength range of the NIR spectrometer is generally 700 nm-2500 nm, and the NIR spectrometer is calibrated, comprising zero calibration and wavelength calibration, before the test begins.

The spectroscopic test is specified as follows: testing and recording the spectral data of each test block using near-infrared spectroscopy, processing the raw spectral data using baseline correction and smoothing filtering to subtract the background, removing the effects of noise and water vapors, analyzing the spectral data to identify the major absorption peaks, and extracting the wavelengths, absorbance, and other eigenvalues for each of the absorption peaks.

The spectral-based multi-classification model is trained using a neural network, specifically, the neural network comprises an input layer, a hidden layer, and an output layer, where the input parameters are the absorption peak features of different test blocks, comprising eigenvalues such as wavelength position and absorbance, and the output parameters are the probabilities of corresponding coal, rock, or gangue categories.

The output layer consists of a fully connected layer and a Softmax layer. For a multi-class classification task with C categories, the fully connected layer produces an output Z={Z₁, Z₂, Z₃, . . . , Z_{C}}, Zᵢ representing the un-normalized score for category i. The Softmax layer then maps the un-normalized scores into a probability distribution, where the probability for each category Pᵢ is computed using the following formula:

$${P}_{i} = \frac{{e}^{{Z}_{i}}}{{\sum }_{j = 1}^{c} {e}^{{Z}_{j}}} ,$$

In this formula, Pᵢ denotes the probability that the input sample x belongs to category i,

$${\sum }_{j = 1}^{c} {e}^{{Z}_{j}}$$

denotes the sum of the exponentiated unnormalized scores across all categories, and C denotes the number of categories, and Z; denotes the unnormalised score of category j.

Wherein, training using neural networks comprises: building a data set from the absorption peak features of different test blocks obtained from testing, comprising wavelength position and absorbance and other feature values and their corresponding coal rock gangue categories, dividing the data set into a training set and a test set, using the cross-entropy loss as a loss function for training on the training set, and evaluating the model's classification accuracy, precision, recall, and F1 scores, etc., using the test set to evaluate the classification accuracy, precision, and F1 score, and the spectral-based multi-classification model is saved after the assessed metrics meet the classification accuracy.

The classification accuracy of the strain-based multi-classification model, the heat-based multi-classification model, and the spectral-based multi-classification model denotes the ratio of correctly classified samples on the test set to the total samples; the classification accuracy is differentiated according to the actual requirements of the site, and the evaluation indexes should be increased when the site has higher requirements for the sorting accuracy of the coal rock gangue.

It should be noted that the strain-based multi-classification model, the heat-based multi-classification model and the spectral-based multi-classification model use the same neural network structure, and the corresponding neural network can be flexibly selected according to the actual situation, and the present example is not specifically limited.

Step 5: Separately using the strain-based multi-classification model, the heat-based multi-classification model, and the spectral-based multi-classification model to sort and identify the mixture of coal, rock, and gangue, fusing the sorting results of the strain-based multi-classification model, the heat-based multi-classification model, and the spectral-based multi-classification model, and determining the final sorting results.

In the present example, in the actual coal, rock and gangue sorting, the 3D non-contact strain monitor, the infrared thermal imager and the near-infrared spectrometer are set up on the conveyor belt to test the coal, rock and gangue mixtures to be sorted, and the coal, rock and gangue are sorted and identified by using the strain-based multi-classification model, the heat-based multi-classification model and the mineral multi-classification model, respectively, and the sorting results of the three multi-classification models are fused at a decision-making level as the final sorting Results.

The actual coal, rock and gangue sorting is as follows: before sorting, using the cutting machine to cut the mined coal, rock and gangue mixture into blocks of the same size, and using the experimental compression machine to compress the blocks before the conveyor belt is transferred.

The 3D non-contact strain monitor is set up on the left side of the conveyor belt and aligned with the test blocks under the experimental compressor, and is used to capture and record the total strain and the corresponding strain value at each moment of time of the test blocks from the beginning of the load to the end of the load. The infrared thermal imager is set up on the right side of the conveyor belt and aligned with the loaded specimens to record the total heat release, the heat release rate and the average temperature of each specimen during the loading process. And, the near-infrared spectrometer is set up on the top of the conveyor belt to test and record the spectral data of each specimen.

The sorting is identified by individually predicting the type of test block using the built strain-based multi-classification model, the built heat-based multi-classification model, and the built spectral-based multi-classification model, respectively, and outputting the corresponding predicted probabilities.

As in the present example, the used strain-based multi-classification model outputs a probability of 40% for fattening coal, 40% for granite, and 20% for lignite gangue; the used heat-based multi-classification model outputs a probability of 40% for fattening coal, 50% for granite, and 10% for lignite gangue; and using the used spectral-based multi-classification model outputs a probability of 24% for fattening coal, and 24% for granite 71% probability, and 5% probability for lignite gangue.

Fusing the sorting results of the strain-based multi-classification model, the heat-based multi-classification model, and the spectral-based multi-classification model, determining the final sorting results, i.e., weighting and averaging the three sorting results, and selecting the category with the largest probability after weighted averaging as the final sorting result, and separately transmitting and transporting the sorted coal and rocks.

In actual coal-rock-gangue sorting, affected by the field environment such as dust, humidity temperature changes, etc., although these multi-classification models have been trained, there may still be uncertainty in practice, in order to reduce the classification error, the present invention fuses the uncertainty predicted by each multi-classification model to determine the final sorting result.

Specifically, a fusion-sorting is specified as: performing a dynamic weighting of the fused model prediction uncertainty, which is quantified by an entropy value, and assigning weights based on the entropy value. For a given sample, the model predicts a category probability distribution p={p₁, p₂, p₃, . . . , p_{c}}, C is the number of categories and Pᵢ is the probability that the model predicts that the sample belongs to category i. The entropy is calculated as:

$$H ( p ) = - {\sum }_{i = 1}^{c} {P}_{i} log ( {P}_{i} ) .$$

When the entropy value is low, it indicates that the model prediction is stable and there is high confidence in the sorting result of the current sample, and higher weights are assigned in the dynamic weighting; when the entropy value is high, it indicates that the model prediction is unstable, and lower weights are assigned in the dynamic weighting.

A weighted average is performed based on the weights, and the category with the highest probability after the weighted average is selected as the final sorting result, and the formula for calculating the weights is as follows:

$${W}_{m} = \frac{1 / {H}_{m}}{{\sum }_{1}^{M} 1 / {H}_{k}} ,$$

where, Hₘ is the entropy value of the m^{th} model to the current sample, M is the total number of classification models, k denotes the k^{th} classification model, Wₘ is the weight of the m^{th} model, and HR is the entropy value of the k^{th} model to the current sample;
Letting the predicted probability of model m be

$${p}_{m} = \{ {p}_{1}^{m} , {p}_{2}^{m} , {p}_{3}^{m} , … , {p}_{c}^{m} \} ,$$

then the weighted final output probability is:

$${p}_{final} = {\sum }_{m = 1}^{M} {W}_{m} * {p}_{m} .$$

As in the present example, assuming that the number of classifications is 3, comprising fat coal, granite and lignite gangue, the predictive probability distribution of the strain-based multi-classification model is p₁={0.7, 0.2, 0.1}, and the calculated entropy value H₁ is 0.610; the predictive probability distribution of the heat-based multi-classification model is p₂={0.4, 0.4, 0.2}, and the calculated entropy value H₂ is 1.029; and the predictive probability distribution of the spectral-based multi-classification model is: p₃={0.8, 0.1, 0.1}, and the calculated entropy value H₃ is 0.500. then the three multi-classification weights of the models are respectively:

$$\begin{matrix}{W}_{1} = \frac{1 / 0.61}{1 / 0.61 + 1 / 1029 + 1 / 0.5} = 0.3 \; 65 , \\ {W}_{2} = \frac{1 / 1.029}{1 / 0.61 + 1 / 1029 + 1 / 0.5} = 0.217 , \\ {W}_{3} = \frac{1 / 0.5}{1 / 0.61 + 1 / 1029 + 1 / 0.5} = 0.418 .\end{matrix}$$

The final fusion prediction is p_{final}=0.365*p₁+0.217*p₂+0.418*p₃, and finally the probability of sorting of the fat coal is 0.6767, the probability of sorting of the granite is 0.2016, and the probability of sorting of the lignite gangue is 0.1217; wherein, the fat coal has the highest probability of sorting and the sample is identified as fat coal.

The sorted coal is transferred to a small conveyor belt corresponding to the coal, wherein the small conveyor belt of coal has a plurality for a total of 6 categories of fat coal, lignite, coking coal, gas fat coal, gas coal, and anthracite, the small conveyor belt of rock has a plurality for a total of 8 categories of granite, gabbro, sandstone, limestone, quartz sandstone, quartzite, plagioclase, and shale, and the small conveyor belt of gangue has a plurality for a total of 2 categories of lignite gangue, and bituminous coal gangue.

The main methods of coal rock gangue sorting comprise manual picking (there are time-consuming and laborious, qualitative judgment inaccurate problems), density difference (there are cumbersome process problems), machine vision (there is a requirement for a complete and clear image of the coal rock gangue, but there are a large number of dust problems in the actual sorting process), in response to the above problems, the present invention selects the three bases of strain, heat, and spectra to be sorted out, and makes use of the joint Different coal rock in the apparent features, physical properties, compositional differences to sorting. These three bases are from different perspectives to achieve the coal rock gangue sorting, in terms of the performance features, taking into account the impact of dust in the sorting environment, the selection of non-contact strain instead of the image for sorting; in terms of physical properties, coal and rock in the thermodynamic properties, molecular structure, mechanical properties of the differences in the compression of the release of heat is different, the choice of release of the temperature of this physical property to be characterized; in the constituent composition In terms of composition, composition is the key to the most essential difference between the reaction object, coal, rock, gangue and its material composition is different, through the spectrum of the composition of quantitative analysis. And the deep decision-making fusion of multiple sorting methods can significantly improve the sorting accuracy of coal, rock and gangue, solve the uncertainty problem caused by a single sorting method, and ensure the accuracy and reliability of the sorting results.

Strain, heat, spectroscopy of these three bases for the combination of sorting, from the apparent features, physical properties, compositional differences in three different perspectives to achieve the coal, rock and gangue sorting; and these three compared to the use of density differences, manual sorting are easy to achieve and simple process, more practical. The starting point of strain-based multi-classification model, heat-based multi-classification model and spectral-based multi-classification model is different level features, easy to achieve, complementary three classification models, deep-level fusion effect is better; and in the fusion of entropy-based uncertainty dynamic weighting, according to the actual performance of the three classification models to flexibly adjust the model weights, adaptability is stronger, classification is more accurate.

### Example 2

The present example provides a coal-rock-gangue sorting system based on multi-source data fusion analysis, comprising:
a sample collection and processing module, configured to: collect different kinds of coal, rock, and gangue, and process them into rock blocks and coal blocks of the same size;
a strain-based multi-classification model building module, configured to: carry out the same loading test on said rock blocks and coal blocks, monitor the strains of the coal blocks and rock blocks, correlate and analyze the different strains with the corresponding categories of coal, rock and gangue, and build a strain-based multi-classification model;
a heat-based multi-classification model building module, configured to: monitor the heat released from said coal blocks and rock blocks during loading, correlate and analyze the heat released under the same size and the same loading conditions with the corresponding coal, rock block and gangue categories, and build a heat-based multi-classification model;
a spectral-based multi-classification model building module, configured to: test and identify the spectral absorption peaks of different categories of coal, rock and gangue, correlate the features of different absorption peaks with the corresponding categories of coal, rock and gangue, and build a spectral-based multi-classification model; and
a fusion sorting module, configured to: sort and identify the mixture of coal, rock and gangue using the strain-based multi-classification model, the heat-based multi-classification model and the spectral-based multi-classification model respectively, fuse the sorting results of the strain-based multi-classification model, the heat-based multi-classification model and the spectral-based multi-classification model, and determine the final sorting results.

### Example 3

It is an object of the present example to provide a computing device, comprising a memory, a processor and a plurality of computer programs stored in the memory and runnable on the processor, when the computer programs are executed by the processor, causing the processor to implement the steps of the method of Example 1.

### Example 4

It is an object of the present example to provide a computer-readable storage medium, a computer-readable storage medium having a plurality of computer programs stored thereon, when the computer programs are executed by the processor, causing the processor to implement the steps of the method of Example 1.

The steps involved in the apparatuses of the above Examples 3 and 4 correspond to the method of Example 1, and the specific implementation mode can be referred to the relevant description part of the Example 1. the term “computer-readable storage medium” should be understood to be non-transitory and comprise a single medium or multiple media comprising one or more sets of instructions; and should also be understood to comprise any medium capable of storing, encoding, or carrying a set of instructions for execution by a processor and causing the processor to perform any of the methodologies of the present invention.

Those skilled in the art will appreciate that the various modules or steps of the invention described above may be implemented using general purpose computer means, alternatively they may be implemented using program code executable by computing means such that they may be stored in memory means for execution by computing means, or fabricated separately as individual integrated circuit modules, or multiple of them may be fabricated as a single integrated circuit module. The present invention is not limited to any particular combination of hardware and software.

The foregoing descriptions are merely preferred embodiments of the present invention but are not intended to limit the present invention. A person skilled in art may make various alterations and variations to the present invention. Any modification, equivalent replacement, or improvement made within the spirit and principle of the present invention shall fall within the protection scope of the present invention.

Although the specific embodiments of the present invention are described above in combination with the accompanying drawings, it is not a limitation on the protection scope of the present invention. Those skilled in the art should understand that on the basis of the technical scheme of the present invention, various modifications or deformations that can be made by those skilled in the art without creative labor are still within the protection scope of the present invention.

1. A coal-rock-gangue sorting method based on multi-source data fusion analysis, comprising:
collecting different categories of coal, rock, and gangue, and processing the collected different categories of coal, rock, and gangue into rock blocks and coal blocks of uniform size;
conducting identical load-bearing tests on the processed rock blocks and coal blocks, monitoring strain responses of the rock blocks and the coal blocks in the tests, correlating different the monitored strain responses with corresponding categories of coal, rock, and gangue, and building a strain-based multi-classification model;
monitoring a heat released from the coal blocks and the rock blocks during the load-bearing tests, correlating emitted heat under identical size and loading conditions with the corresponding categories of coal, rock, and gangue, and building a heat-based multi-classification model;
performing a spectral analysis on the different categories of coal, rock, and gangue to identify spectral absorption peak features, correlating the identified spectral absorption peak features with the corresponding categories of coal, rock, and gangue, and building a spectral-based multi-classification model; and
employing the strain-based, heat-based, and spectral-based multi-classification models to independently classify coal-rock-gangue mixtures, and by fusing sorting results of the strain-based, heat-based, and spectral-based multi-classification models, determining and outputting a final sorting result;
wherein, determining and outputting the final sorting result by fusing the sorting results of the strain-based, heat-based, and spectral-based multi-classification models, specifically comprising: applying a weighted averaging method to three the sorting results, selecting a category with the highest probability after the weighted averaging as the final sorting result, and separately conveying and transporting the separated coal and rock;
a process of the weighting is a dynamic weighting based on an uncertainty of prediction of a fusion model, the uncertainty is quantified by using an entropy value, and weights are assigned according to entropy values; the entropy value is computed using the following formula:
$$H ( p ) = - {\sum }_{i = 1}^{c} {P}_{i} log ( {P}_{i} ) ,$$
wherein, p denotes the probability distribution of the categories predicted by the model, C denotes a total number of the categories, and Pᵢ denotes a probability that the model classifies a sample into category i.

2. The coal-rock-gangue sorting method based on multi-source data fusion analysis according to claim 1, wherein the strain-based multi-classification model is trained using a first neural network, wherein input parameters comprise a total strain of the blocks and strain values recorded at different time intervals, while an output parameter is a probability distribution of a corresponding type of coal, rock, or gangue.

3. The coal-rock-gangue sorting method based on multi-source data fusion analysis according to claim 1, wherein the heat-based multi-classification model is trained using a second neural network, wherein input parameters comprise a total heat released, a heat release rate, and an average temperature of the blocks under the identical size and load-bearing conditions, while an output parameter is the probability distribution of the corresponding type of coal, rock, or gangue.

4. The coal-rock-gangue sorting method based on multi-source data fusion analysis according to claim 1, wherein the spectral-based multi-classification model is trained using a third neural network, wherein input parameters comprise the absorption peak features from different blocks, while an output parameter is the probability distribution of the corresponding type of coal, rock, or gangue.

5. The coal-rock-gangue sorting method based on multi-source data fusion analysis according to claim 1, wherein applying the weighted averaging method based on the assigned weights, and selecting the category with the highest probability after weighted averaging is selected as the final sorting result; the calculation formula of the weight Wₘ is as follows:
$${W}_{m} = \frac{1 / {H}_{m}}{{\sum }_{1}^{M} 1 / {H}_{k}} ,$$
wherein, Hₘ denotes the entropy value of the m^{th} model for a current sample, M denotes a total number of classification models, k denotes the k^{th} classification model, Wₘ denotes the weight assigned to the m^{th} model, and Hₖ denotes the entropy value of the k^{th} model for the current sample.

6. A coal-rock-gangue sorting system based on multi-source data fusion analysis, comprising:
a sample collection and processing module, configured to: collect different categories of coal, rock, and gangue and process the collected coal, rock, and gangue into rock blocks and coal blocks of uniform size;
a strain-based multi-classification model building module, configured to performs identical load-bearing tests, monitor strain responses of the coal blocks and the rock blocks, and build a strain-based multi-classification model;
a heat-based multi-classification model building module, configured to monitor heat released from the coal blocks and the rock blocks during the load-bearing tests and build a heat-based multi-classification model;
a spectral-based multi-classification model building module, configured to analyze spectral absorption peak features of different coal, rock, and gangue samples and build a spectral-based multi-classification model; and
a fusion sorting module, configured to integrate sorting results from the strain-based, heat-based, and spectral-based multi-classification models to determine a final sorting result;
wherein, determining and outputting the final sorting result by fusing the sorting results of the strain-based, heat-based, and spectral-based multi-classification models, specifically comprising: applying a weighted averaging method to three the sorting results, selecting a category with the highest probability after the weighted averaging as the final sorting result, and separately conveying and transporting the separated coal and rock;
a process of the weighting is a dynamic weighting based on an uncertainty of prediction of a fusion model, the uncertainty is quantified by using an entropy value, and weights are assigned according to entropy values; the entropy value is computed using the following formula:
$$H ( p ) = - {\sum }_{i = 1}^{c} {P}_{i} log ( {P}_{i} ) ,$$
wherein, p denotes the probability distribution of the categories predicted by the model, C denotes a total number of the categories, and Pᵢ denotes a probability that the model classifies a sample into category i.

7. A computer-readable storage medium, having computer programs stored thereon, wherein when the computer programs are executed by a processor, causing the processor to implement steps of a coal-rock-gangue sorting method based on multi-source data fusion analysis according to claim 1.

8. A computing device, comprising a memory, a processor and computer programs stored in the memory and runnable on the processor, when the computer programs are executed by the processor, causing the processor to implement steps of a coal-rock-gangue sorting method based on multi-source data fusion analysis according to claim 1.
